(19) United States
(12) Patent Application Publication
(10) Pub. No.: US 2005/0266049 A1
(43) Pub. Date: Dec. 1, 2005

(54) Method for the covering of food with polyene antifungal compositions

(75) Inventors: Martin Johannes Maria Warmerdam, Ruswijk (NL); Ben Rudolf De Haan, Voorburg (NL)

Correspondence Address: MORRISON & FOERSTER LLP, 3811 VALLEY CENTRE DRIVE, SUITE 500, SAN DIEGO, CA, 92130-2332, US

(73) Assignee: D S M IP Assets B.V., Heerlen (NL)

(21) Appl. No.: 10/523,881

(22) PCT Filed: Aug. 6, 2003
(86) PCT No.: PCT/EP03/08768

§ 371 Date: Feb. 7, 2005

(30) Foreign Application Priority Data
Aug. 6, 2002 (EP) 02102100.1

(51) Int. Cl. A61K 47/00; A23C 9/12; A23L 1/315

(52) U.S. Cl. 424/439; 426/34; 426/55

(57) ABSTRACT

The present invention relates to a food product (such as sausage or cheese) having a mould-like appearance which has a coating of a powder which gives a mould-like appearance, whereby the powder comprises a polyene antifungal compound. (such as natamycin).

### FIELD OF THE INVENTION

The present invention relates to a process to prevent mould growth on a food product comprising treating the product with a flour-like powder to obtain a mould-like appearance. Products treated by this process, e.g. food products such as sausages and cheeses, are also included in this invention.

### BACKGROUND OF THE INVENTION

For more than 30 years natamycin has been used to prevent fungal growth on cheeses and sausages.

Cheeses are treated by immersion in a suspension of natamycin in water or covered by an emulsion of a polymer in water, mostly polyvinylacetate. Sausages are mainly treated by immersion in or by spray coating with a suspension of natamycin in water. Aqueous suspensions for immersion treatments usually contain 0.1% to 0.2% w/v of natamycin. Polymer emulsions for coating purposes usually contain 0.01% to 0.05% w/v of natamycin.

These treatments are mostly effective in preventing growth of moulds or yeasts on the treated products. The solubility of natamycin in water is 30-50 ppm (Brik, H.; “Natamycin” Analytical Profiles of Drug Substances 10, 513-561 (1981)), while its Minimal Inhibitory Concentration (MIC) for most foodborn fungi is less than 10 ppm. Under normal conditions the amount of dissolved natamycin will be sufficient to protect food products such as cheeses or sausages against fungal spoilage. Slow dissolution from the natamycin crystals will even protect a treated product for a longer period of time, which is one of the most important advantages of polyene fungicides such as natamycin.

In the case of a fungicide which has a low solubility and which only has antifungal activity in solution such as natamycin, various factors will determine the final fungicidal effect. These include the dissolution of the fungicide, the diffusion of the dissolved fungicide to the site of action and the degradation of the fungicide. Dissolved polyene fungicides may be degraded by several ways, e.g. by hydrolysis, by the action of light or by its irreversible binding to the fungal cell membrane leading to lysis of the fungal cell. In most cases the efficacy of polyene fungicides, such as natamycin, will not be limited because elimination of dissolved natamycin will be compensated sufficiently by dissolution of undissolved natamycin and by diffusion of dissolved natamycin to the site of action. Furthermore, the MIC value for most fungal species is below the solubility of the fungicide.

Mould protection in the meat industry can normally be obtained by soaking the empty sausage casing or dipping the whole sausage in a suspension of polyene fungicides, e.g. natamycin.

Usually aqueous suspensions for such immersion treatments contain 0.05% to 0.1% w/v of natamycin in combination with 8-10% (w/v) NaCl.

In the dried fermented sausage industry, traditional surface ripening is obtained by addition of moulds.

In some cases, the ripened and visibly mouldy sausages are washed before selling.

When the product is sold as a traditional ripened and mouldy sausage, the sausage may be covered with a mould-like, food grade powder like starch or flour.

Adhesion of the mould-like powder to the product is carried out by battering, by special equipment or by methods like electrostatical adhesion.

The sausages are further packed in vacuum, MAP or in permeable plastic bags.

However, moulds may start to grow again during storage due to remaining mould spores and/or mycelium. This may result in an undesired appearance of the sausage.

There is therefore a need for mould-free sausages which can be stored for at least two months, in particular sausages that appear as if they have mould (but in fact do not).

### DESCRIPTION OF THE INVENTION

The present invention relates to a food product which has a coating of a powder comprising an antifungal compound, wherein the powder provides a mould-like appearance.

The present invention also relates to a food product which has a coating of a powder comprising an antifungal compound, wherein the coating comprises 0.005-10 g/dm², preferably 0.01-7 g/dm² of powder on the surface area of the food product. According to another embodiment of the present invention a food product is provided which has a coating of a powder whereby coating of the powder is visually (by the eye) as an uniform coating.

The present invention discloses a process for coating a food product which comprises adding a dry powder to the surface area of the food product, wherein the dry powder comprises an antifungal compound.

The present invention relates to a process comprising adding a powder together with a polyene fungicide, e.g. natamycin, preferably to obtain a mould-like appearance to the surface of a foodstuff such as a sausage. The present invention also relates to a composition comprising powder and a polyene fungicide, which is useful for e.g. combatting moulds and yeasts on foodstuff such as a fermented sausage. Preferably, this composition can be applied to sausages without the need to adjust or change the established production methods and processes.

Unexpectedly it has been found that on the surface of a fatty dried fermented sausage the activity of polyene fungicides, such as natamycin, when added in dry state can be sufficient to avoid undesired mould growth during storage.

When natamycin is applied, before visible moulds are present, a washing step may be avoided in some cases. Using the composition of the invention comprising a powder such as flour, for example rice flour, together with a polyene fungicide, such as natamycin, a sausage with a mould-like appearance and which is free of undesired mould formation during storage can be obtained.

The polyene fungicide, preferably natamycin, is applied together with the powder in dry form without the used of water as carrier or solvent. The present invention provides sausages that can be stored at 12° C. for at least 60 days and which are free of visible mould growth on the coating. The sausages can be stored in open air and thus a plastic coating or storage under special conditions, such as oxygen free conditions, are not needed. A sausage having a mould-like appearance is a sausage that has been coated or treated with a powder, like flour or starch, to obtain a mould-like appearance.

The invention also relates to methods of preparing antifungal compositions containing an antifungal compound of the polyene type, preferably natamycin, and a powder with a mould-like appearance (when applied on a foodstuff) like flour or starch, preferably rice flour. Said compositions comprise an amount of polyene fungicides of preferably 10-5000 ppm natamycin, more preferably 50-2000 ppm and most preferably 100-1500 ppm. In general 0.005-10 mg of natamycin per dm2 of surface area of the food is used, more preferably 0.01-7 mg natamycin per dm2 and most preferably 0.1-5 mg natamycin per dm2 is used.

In a preferred aspect, the composition of the invention is prepared by dry mixing the ingredients. Said composition has an activity towards fungi. Since only the dissolved fraction of a polyene fungicide has antifungal activity, it is surprising that said dry compositions have a remarkable activity against moulds and yeast when used on sausages.

The polyene fungicide may be, for example, natamycin, lucensomycin, nystatin or amphotericin B. Preferred polyenes are natamycin and amphotericin B, most preferred is natamycin. Also crystal forms, derivates and salts of polyene fungicides, which are for example described in European Patent Application EP-A-865738, may be used. Examples of possible crystal forms are α-natamycin, δ-natamycin and γ-natamycin. Examples of possible solvates are methanol and ethanol solvates of polyene fungicides. Examples of possible salts are calcium and barium salts of e.g. natamycin.

The powder can be composed of any food grade covering material and is mostly based on starch or flour or a combination thereof and has a food grade origin such as wheat, barley, corn, rice, rye, soy, oat and breadcrumbs amongst others. The starch or flour may be pretreated or reformulated, e.g. chemically, enzymatically or physically treated, such as milled, if required.

The composition of the invention may also comprise other functional additives such as proteins, gums, salts, leavening agents, coloring agents and seasoning amongst others.

The color of the covering coating on the foodstuff can be any desired colour from clear white to dark brown or even black.

Colour variations can be introduced by changing the powder grade, powder particle size, flour type, or by roasting the used powders and/or introducing colouring pigments.

The compositions according to the invention may be used in one or more steps on the fermented sausage. For example, two steps may be carried out by first pre-treating the foodstuff followed by the main treatment. All treatments are preferably done by means of dipping the foodstuff in the dry composition containing starch or flour and the polyene fungicide, for example dipping in combination with electrostatic adhesion. Food products such as cheeses and sausages, preferably sausages are treated in this way. Whole or part of cheeses can be used in this method. The present invention provides sausages that can be stored at 12° C. for at least 60 days and which are free of visible mould growth on the coating.

The food products used in the invention need minimal dimensions to obtain homogeneous coating characteristics and mould-like appearance. The SSA, specific surface area of the foodstuff, is preferably equal or less than 1 m2/kg to obtain homogeneous covered food products with sufficient amounts of coating material.

The amount of powder material needed to obtain a regularly covered food will preferably be in a range of 0.005-10 g/dm², more preferably in a range of 0.01-7 g/dm² and most preferably 0.1-5 g/dm² of powder on surface area of the food used.

### EXAMPLES

### Example 1

To standard rice flour (grade powder), natamycin was added to a final concentration of 1000 ppm. This composition was mixed until homogeneous.

The rice flour/natamycin mixture was used as coating by dipping (electrostatically) washed (and dried) fermented salami type sausages in the mixture.

The amount of flour mixture used was 10 kg per ton of sausage.

The sausages were stored at 85% RH (relative humidity) and 12° C. and checked for mould growth. Washed (and dried) sausages were treated with only rice flour, of the same type as used for the mixture and stored as controls. The trial resulted in the following results:

| | Visible moulds | | | |
|---|---|---|---|---|
| — | After 0 days | After 10 days | After 30 days | After 60 days |
| Reference salami | − | +/− | ++ | ++ |
| (rice flour treated) | | | | |
| Invention salami | − | − | − | − |
| (rice flour/natamycin) | | | | |
| treated | | | | |

− no mould visible
+/− hardly any mould visible
+ mould visible
++ many mould spots visible

Whole cheeses or part of cheeses can be used in this method as well.

1. A food product which has a coating of a powder comprising an antifungal compound, wherein the powder provides a mould-like appearance.

2. A food product which has a coating of a powder comprising an antifungal compound, wherein the coating comprises 0.005-10 g/dm² of powder on the surface area of the food product.

3. The food product of claim 1, wherein the food product is a cheese or sausage.

4. The food product of claim 1, wherein the powder comprises flour or starch.

5. The food product of claim 1, wherein the antifungal compound is a polyene fungicide.

6. The food product of claim 5, wherein the polyene fungicide is natamycin.

7. The food product of claim 6, wherein the coating provides 0.005-10 mg of natamycin per dm of surface area of the food product.

8. The food product of claim 1, wherein the food product can be stored for 60 days at 12° C. in open air conditions with no visible mould growth on the coating.

9. A method for coating a food product which comprises adding a dry powder to the surface area of a food product, wherein the dry powder comprises an antifungal compound.

10. The method of claim 9 whereby 0.005-10 g/dm² of dry powder is added per dm² of surface area of the food product.

11. The method of claim 9 whereby dry powder is added to provide a mould-like appearance.

12. (canceled)

13. The food product of claim 2 wherein the coating comprises 0.01-7 g/dm² of powder.
